(19) United States
(12) Patent Application Publication
(10) Pub. No.: US 2025/0017526 A1
(43) Pub. Date: Jan. 16, 2025

(54) METHOD FOR SCREENING POTENTIAL ANTIDEPRESSANT AND PSYCHOTROPIC SUBSTANCES WITH SLEEP RELATED MEASURES

(71) Applicants: Barak Avrum Pearlmutter, Dublin (IE); Jonathan Vittorio Giordano, Laghey (IE)

(72) Inventors: Barak Avrum Pearlmutter, Dublin (IE); Jonathan Vittorio Giordano, Laghey (IE)

(21) Appl. No.: 18/749,287

(22) Filed: Jun. 20, 2024

Related U.S. Application Data
(60) Provisional application No. 63/521,138, filed on Jun. 20, 2023.

(51) Int. Cl. A61B 5/00; A61B 5/0205

(52) U.S. Cl. CPC A61B 5/4848; A61B 5/0205; A61B 5/4812; A61B 5/4815; A61B 2503/40; A61B 2503/42

(57) ABSTRACT

This inventive method leverages physiological measures of sleep, in particular those related to Random Eye Movement Sleep (REM), in order to screen for substances or procedures which may have anti-depressive properties. This method is intended for use as an assay with appropriate controls, and discrete experimental periods of baseline, treatment, and recovery. Three principal criteria are used to determine the consideration of an experimental treatment as an antidepressant candidate: significant and persistent reduction in REM Sleep percentage during the treatment period and evidence for maintaining REM pressure into the recovery recovery period. Additional measures are used to aid with selection including, increase in the Delta Ratio, increase REM Density, increase in REM latency, and change in Ultradian Rhythm length.

### RELATED APPLICATIONS

This application claims the benefit of U.S. Provisional Patent Application No. 63/521,138 filed Jun. 20, 2023, some of the contents of which are hereby incorporated herein.

### FIELD OF THE INVENTION

The present invention relates to the field of drug screening, in particular but not limited to, the identification of prospective anti-depressant and psychotropic substances through the evaluation of sleep effects on animal models or individuals.

### BACKGROUND

Depression is a mental health disorder characterized by persistent negative feelings such as sadness, hopelessness, and anhedonia, and its effects can range from mild disability to a potential cause of suicide. According to the World Health Organization (WHO), depression is the leading cause of disability worldwide and affects an estimated 300 million people or 4.4% of the global population. Depression is also a major contributor to suicide, which in 2015 accounted for nearly 1.5% of all deaths, and was the second largest cause of death for persons aged 15-29 (World Health Organization, 2017).

Antidepressant drugs are a principal treatment method for depression. Typical antide-pressants include Monoamine Oxidase Inhibitors (MAOIs), Tricyclic Antidepressants (TCAs), and Selective Serotonin Reuptake Inhibitors (SSRIs). However, antidepressants can cause unwanted side-effects, and these will affect the majority of users (Bet et al., 2013). Side effects include dry mouth, weight gain or loss of appetite, headaches, disturbed sleep and insomnia, sexual dysfunction, profuse sweating, drowsiness, constipation, and hypertension (Uher et al., 2009). Antidepressants can also directly cause death through serotonin syndrome (Cleveland Clinic, 2023). Furthermore, treatment with antidepressants requires a titration period and takes at least 3-4 weeks before an improvement in condition is clinically noticeable for most users (Nielsen et al., 2000). Most worry-ingly, there is mounting evidence of the potential for antidepressants to increase the risk of suicidal behavior, especially for those below the age of 25 (Levenson and Holland, 2006; Hengartner, 2017). Quitting antidepressants also requires a tapering period of several weeks to months in order to avoid withdrawal symptoms. Shorter term tapering is not advised and can leave users dealing with undesirable side-effects while also waiting to attempt another drug treatment (Horowitz and Taylor, 2019). Some antidepressants require strict dietary awareness, and generally antidepressant users must be conscious of other drugs they take, such as alcohol, due to drug interactions (Koski et al., 2005; Flockhart, 2012; Menkes and Herxheimer, 2014).

Besides these shortcomings of antidepressant medication, treating depression is accom-panied with the the additional challenge of utilizing, let alone identifying, potential bio-markers to measure disease level and quantify treatment effect size, and this is typical for most neuro-psychiatric disorders (Strawbridge et al., 2017). There is no widespread clinical usage of biomarkers to determine the best treatment type or assess treatment efficacy when it comes to antidepressant medications. Improvement results are determined by self-report which can be a limiting factor especially when treatments take on the order of weeks to months to realize if they are ultimately effective.

And yet, antidepressants remain one of the primary recourses for alleviating depressive symptoms. In 2018, it was estimated that over 13% of adults in the United States of America (USA) were on antidepressants and in 2020 over 12% of medical card holders in the Republic of Ireland were prescribed antidepressants (Brody and Gu, 2020; McCool et al., 2021). For 29 Organisation for Economic Co-operation and Development (OECD) countries which had published pharmaceutical data relating to antidepressant consumption in 2020, 6.7% of the combined population was on antidepressants. Among the top 5 consuming countries consisting of Iceland, Portugal, Australia, Canada, and Sweden, 12.6% of the population were on antidepressants (OECD, 2023).

Given the high usage of antidepressants in spite of their drawbacks reveals their necessity. It is clear that improved psychotropic medication for depression with better symptom alleviation, and fewer and less severe side effects, would be of great benefit to millions of people. However, the process for discovering potential medications and evaluating their efficacy and safety is long and costly. To achieve faster discoveries of better medications requires an improved method for utilizing bio-markers and physiological measures to screen potential substances of benefit, for conditions such as but not limited to, depression. The behavioral state of sleep is a window for leveraging such measures to achieve this screening.

For most mammals, sleep is a multi-staged behavioral state of apparent quiescence. In humans it can be divided into two main stages: Random Eye Movement Sleep (REM), also known as paradoxical sleep, and Non-Random Eye Movement Sleep (NREM). Although there have been advances in our understanding of sleep, it remains unclear what the function or functions of each stage are, though there are many theories (McNamara, 2019). Deprivation experiments where specific sleep stages are halted or disturbed are one method used by researchers to understand their respective importance.

The 1960's and 1970's saw a flourish of experiments exploring the effects of REM Sleep Deprivation (REMD) on rodents and other animals (Dement, 1965b; Morden et al., 1967; Albert et al., 1970; Stern et al., 1971; Mendelson et al., 1974). One of the observed effects of REMD was heightened behavioral activity, including increased response rates to, and lower thresholds for, rewards (Steiner and Ellman, 1972). In other words, REMD appeared to have antidepressant properties.

While there were initial concerns of the safety of REMD by physical arousal on humans, largely by the mid 1960s the idea that REMD was nocive to the human psyche was no longer supported (Dement, 1960; Dement and Fisher, 1963; Dement, 1964; Fisher, 1965; Dement, 1965a; Vogel, 1975). Further investigations even failed to produce observations of psychological harm of REMD on vulnerable patients, such as 5 schizophrenics who were REM deprived for one week, and 9 severely depressed patients who were REM deprived for 1-2 weeks (Vogel and Traub, 1968a,b; Vogel et al., 1968). A more modern study also found no psychological disturbances in 30 participants, 20 of whom were depressed (Cartwright et al., 2003). Most importantly, in addition to no having apparent and significant negative psychological consequences, several studies have demonstrated REMD to have anti-depressive effects on humans (Vogel and Traub, 1968a,b; Vogel et al., 1975; Cartwright et al., 2003).

The most notable of these studies was led by Gerald W. Vogel from 1972-1975 (Vogel et al., 1975). Encouraged by promising results from pilot studies, Vogel decided to test the anti-depressive effectiveness of REMD by awakenings (Vogel et al., 1968; Vogel and Traub, 1968a). In their 1975 study, Vogel et al. effected REMD on more than 50 hospitalized depressed patients. Patients were independently verified to have moderate to severe depression by two psychiatrists, and absent of other conditions such as substance abuse issues or schizophrenia. In the patient group consisting of individuals with endogenous depression, a sub-group where the cause is attributed to internal stressors (genetics, biological phenomenon, etc.), 17 of 34 patients (50%) responded to the REMD treatment and were discharged from the hospital. Of the 17 patients who responded positively, 13 patients (76%) maintained their mental health for at least 6 months after treatment. Of the patients who did not respond to the REMD treatment, 9 then completed as a follow up treatment a (at minimum) 4 week course of imipramine, a tricyclic antidepressant. It should be noted 11 patients initially elected for this follow up treatment, but 2 dropped out during the treatment course. Only 1 of the 9 patients (11%) who completed 4 weeks of treatment responded to the drug (Vogel et al., 1975).

Incidentally, imipramine is known to cause significant and persistent REMD (Dunleavy et al., 1972). The unusually low success rate of the imipramine treatment after failure to re-spond to REMD by arousal lends support to the hypothesis that REMD is largely responsible for imipramine's antidepressant effect (Vogel et al., 1975; Vogel, 1983). In fact, many antidepres-sants have REM suppressing effects suggesting that this may be the main mechanism for their anti-depressive properties (Vogel et al., 1990).

Currently, the mechanisms by which antidepressants work remain a mystery. Even in-fluential theories such as the serotonin hypothesis of depression, upon which selective serotonin reuptake inhibitors (SSRIs) are theoretically based, lack confirmation in spite of the numerous in-vestigations that have been carried out (Moncrieff et al., 2022). However, to our knowledge, every drug which satisfies 3 REMD treatment criteria has been shown to have anti-depressive effects. First, the level of REMD must be significant, decreasing total REM sleep by roughly 50% or more of the baseline REM sleep amount. Second, this reduction in REMD is largely sustained over time, though not necessarily to the initial level of reduction. In humans this time scale is on the order of several weeks. And finally, halting REMD, regardless of its method of induction, results in an REM rebound, whereby there is a significant increase in REM sleep compared to baseline. In other words, REM pressure, the internal drive for the REM sleep, is maintained or increased during the REMD period (Vogel et al., 1990).

An important observation that Vogel et al. noted in their research is that REM rebound was more strongly seen in depressed individuals who responded to treatment, and that individuals who did not respond showed weaker if any rebound (Vogel et al., 1968; Vogel and Traub, 1968a). Measuring REM rebound propensity is therefore a potential biomarker for quantifying “depression level” for a subgroup of depression and this can be leveraged for predicting likelihood of treatment outcome, for forecasting treatment efficacy, and for evaluating new potential treatments.

Consequently, there are 2 principle issues to highlight. First, the drug discovery process, in particular for antidepressants, relies heavily on animal models. Mice and rats are especially used for this type of research, and are subject to various stressors. These include social isolation, tail suspension tests, and forced swim tests. This poses a labor problem, as constantly stressing animals is an intensive process for researchers (Willner, 1997). Secondly it is also an ethical quandary, and in recent times genetic modifications in mice are being pursued to better replicate depressive symptoms such as anhedonia which are even more ethically concerning (Cryan and Holmes, 2005). Therefore, a novel method of treatment screening is needed which is both less labor intensive and has fewer potential ethical issues.

With this understanding of REMD as a treatment, we will now examine how our method carries the potential to be a novel assay for prospective depression treatments.

### OBJECTS OF THE INVENTION

It is an object of this invention to provide a method for clinical assays for antidepressant and psychotropic substances which is based on physiological measures and is both less labor intensive and has fewer potential ethical issues.

### SUMMARY OF THE INVENTION

In order to overcome obstacles associated with the screening of potential treatments for depression and other neuro-psychiatric disorders, we have invented a method of using physiological measures of sleep in an assay. This is carried out through the collection and analysis of sleep mea-sures of animal models or human participants during a baseline, treatment, and recovery period. Tested substances are only administered during the treatment period, while a baseline period serves to establish normal measures before intervention, and a recovery period to capture the discontinua-tion effect of the intervention. The principal measures for consideration are (1) the change in REM Sleep Percentage (REM %) of Total Sleep Time (TST) between baseline and treatment, (2) the change in REM % between baseline and recovery, and (3) the trend of change in REM % during the treatment period. Additional measure for consideration include alterations to REM latency, REM density, and Delta ratio. Successful candidate substances will be selected based upon assessment of these measures.

### BRIEF DESCRIPTION OF THE DRAWINGS

For a more complete understanding of the invention, reference is made to the following brief descriptions accompanying drawings in which:

FIG. 1 shows an example plot of the REM Percentage of Total Sleep Time (TST) for a given treatment, whether it be procedure, method, or pharmacological, which satisfies the 3 known criteria for REMD to be effective as an anti-depressant. Shown are 2 baseline days prior to the treatment, 5 days of treatment, followed by 2 days of post-treatment monitoring. There is an obvious lowering of the REM percentage during the treatment, and a clear REM rebound post-treatment.

It is noted that the timescale presented in FIG. 1 is not necessarily representative of an actual treatment or screening test, but it used to clarify the expected trend over time. Similarly, the percentages and counts of REM sleep provided are for demonstration of expected trends.

FIG. 2 show possible methods for observing animals, in particular to measure their sleep stages. The rodent on the left demonstrates contact methods of observation, and the rodent on the right non-contact methods.

FIG. 3 shows the average REM sleep time percentage for each experimental group during baseline, treatment, and recovery periods. This figure could be representative of a drug trial screening substances A, B, and C in an homogenous animal population or group of individuals. The control represents a group of this population receiving no treatment. Groups A, B, C, named after their respective test substances, display differences in changes to REM percentage throughout the trial. Group A shows minor, but non-significant changes in REM percentage during and between all testing periods. In comparison, Group B demonstrates a significant reduction in REM percentage during treatment, but not between baseline and recovery periods. This indicates that substance B while being a significant REM sleep reducer, does maintain or increase REM sleep pressure during treatment. Finally, Group C demonstrates a significant reduction in REM sleep during treatment, as well as a clear REM sleep rebound during the recovery period. This indicates that substance C is a significant REM reducing agent, while maintaining or increasing REM pressure. Of all substances, only substance C would pass the screening test of possible anti-depressive agent, barring other information such as observed side-effects.

FIG. 4 demonstrates a possible set-up for an example screening of a homogenous rodent population divided into three testing groups, A, B, and C and a Control group. Each group contains N=1, 2, . . . , X testing animals as denoted in the figure. In this example, a rodent with contact sensors [16] is presented as the animal model and method of monitoring, with each animal having its own enclosure [19]. For clarity, this example shows one animal per enclosure but in practice having multiple animals per enclosure is not precluded. Each animal is labeled first by their respective group letter, i.e., A, B, or C, followed by respective number in sequential order, i.e., 1, 2, . . . , X. Such a set-up as shown in the figure may allow for the testing of at least twos substances, with one group serving as a control. For example, Group A might serve as a control and Group B may serve to test Substance B while Group C is used to test Substance C. If behavioral and physiological measures of a given animal model are well known, a control group may be unnecessary, but this is not recommended as it does not alllow testers to control for any local or temporal changes that may arise during testing.

FIG. 5 shows a method for real-time classification of a signal.

FIG. 6 shows a EOG and head worn accelerometer signals for a Wake state.

FIG. 7 shows EOG and head worn accelerometer signals for a NREM sleep state.

FIG. 8 shows EOG and head won accelerometer signals for an REM sleep state.

### DETAILED DESCRIPTION AND EXAMPLE EMBODIMENT

There are many possible arrangements for utilizing our method, and we will explain in detail one potential embodiment here. For clarity, this serves as a set example with the under-standing that it is only one out of a variety of configurations and possible modalities. Therefore, it is recognized that this example is chosen in order to illustrate the spirit of the invention, not to limit its scope to one specific embodiment.

Suppose it may be of question whether a substance or set of substances have antidepressive properties and/or might serve as a beneficial psychotropic medication. Typically such substances are screened through animal models before testing on human participants occurs. For this exam-ple, we follow in these general footsteps, but this does not preclude the method from also being utilized in human volunteers, even before animal trials. Instead of focusing on animal behavioral responses which most generally describe resilience and drives and require intensive human labor and monitoring, our method accounts for changes in physiological measures of sleep before, during, and after treatment with the substances in question to determine its psychotropic potential.

To begin, we start with an appropriate animal model and set up such as demonstrated in FIG. 2. Here there is a suitable enclosure [19], and two healthy example rodents. It is noted that other types of healthy and suitable animals may be used. Typically when recording detailed sleep measures in rodents, a sensor cap [15] is fitted, as seen on the left of FIG. 2 with the rodent with contact sensors [16]. This cap provides the attachment and housing for various sensors such as EMG, EEG, and/or intracranial electrodes. Information from the sensor cap [15] is then transmitted by cable [14] to an appropriate recording and analyzing device. This method is inconvenient for a variety of reasons including labor (surgery must often be performed), risk of infection, and undesirable influences on rodent behavior and movement. Using wireless methods instead of a cable [14] can mitigate some of these caveats, but it not a complete solution. On the right a free rodent [17] is shown with an improved method of monitoring. The term “free” here is used to denote the fact that the animal is not tethered or required to carry any equipment, and so may move and act most naturally. This method utilizes a non-contact sensor [18] or set of them. These can include, but are not limited to, video cameras, thermal imaging, microphones, and radar. Non-constant contact sensors can also be used, such as an electronic sensor mat placed on the bottom of the enclosure [19]. Physiological information can be interpreted from the data collected by these non-contact and non-constant contact sensors to thereby determine the wake, sleep, and specific sleep state of the animals without the need for surgery and disturbance to the rodents. It is noted these technologies extend to other animals, including humans. Although two rodent examples are shown here with different monitoring methods, for economic and analysis purposes it may be preferred to utilize only one method. This does not preclude using a few subjects with a more costly and invasive, but more accurate monitoring technique such as the sensor cap [15] on some subjects as a sanity or accuracy check.

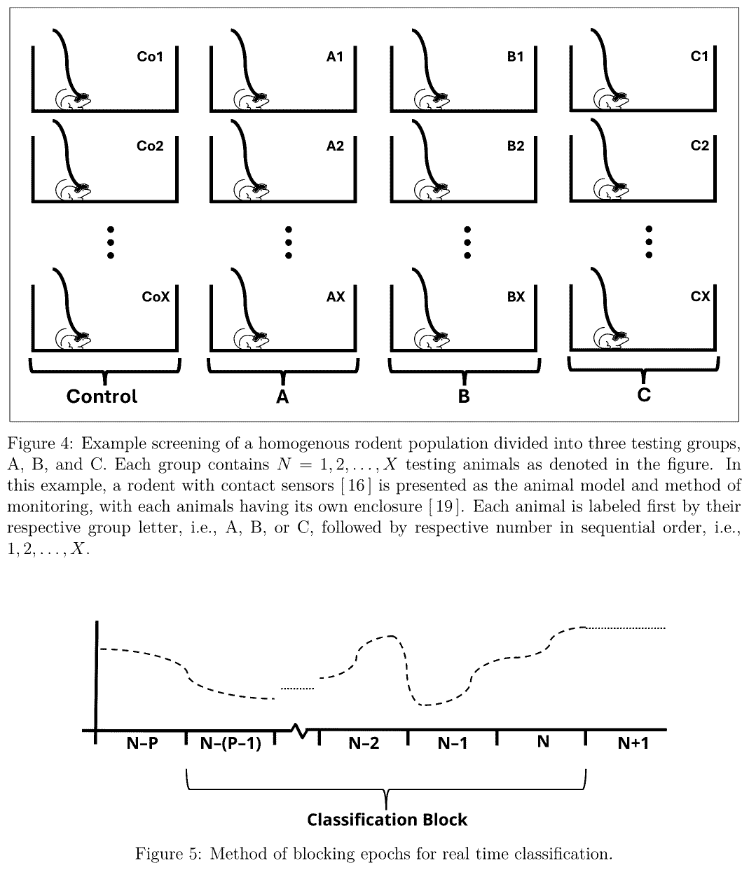

Given that there is variation in the real-world, such as differences in genetics even in a homogenous population, it is best to confirm the success of a substance over a representative group. Therefore, as is presented in this example, it is advised screening occurs over a suitable number of subjects, such as demonstrated in FIG. 4. In essence, this figure demonstrates a multiplication and a grouping of the unit described in FIG. 2. Each unit consists of a single rodent, monitoring method, and enclosure, but the units number in quantity such that there is a suitable group size for testing multiple substances and having a control group. Explicitly, a single unit here consists of an enclosure [19] with a rodent with contact sensors [16] and, though not seen, the necessary equipment such as a computer for collecting and processing data. The units are divided into three testing groups, A, B, and C, each for a respective substance or combination thereof, and a Control group. Every group has an equal number of units (i.e., subjects) numbered from N=1, 2, . . . , X where ‘X’ is a suitable group size, for example ‘10’. Each unit, and therefore each subject, is labeled first by their respective group letter, i.e., ‘A’, ‘B’, or ‘C’, followed by respective number in sequential order, i.e., 1, 2, . . . , X. The Control group is prefixed by ‘Co’. It is noted that if the behavioral and physiological measures of a given animal model are well known, a control group may not be necessary, however this is not recommended as it will not allow for controlling any local or temporal changes that may arise during testing.

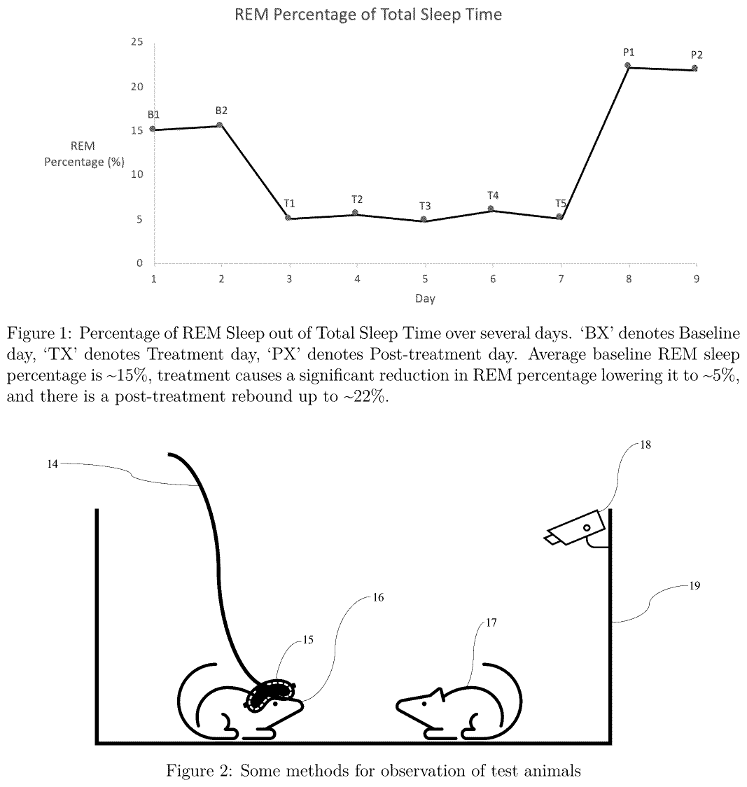

Given substances A, B, and C for which it is desired to screen for antidepressive or psy-chotropic potential, and an appropriate testing set-up, such as described in FIG. 4 with organized groups, we will now describe the method. First, data will be gathered during baseline period. Dur-ing this time, no changes of any kind, experimental or otherwise, should occur. All animals should already be accustomed to their environments, and if this is not already done, such as animals being recently moved to new enclosures, this habituation time should be accounted for and data during this period should be excluded in the analysis. Similarly in humans, the “First Night Effect” is well established and should be taken into account for human trials. The baseline monitoring should occur over a period of at least 2 days, as demonstrated in FIG. 1, in order to observe a stable approximation for typical sleep measures among the subjects. Such measures may include, but are not limited to the following:
1. Total Sleep Time (TST)—The amount of total sleep of a determined duration (i.e. a 24 hour period)
2. REM Sleep Percentage (REM %)—Percentage of Random Eye Movement Sleep of the TST
3. NREM Sleep Percentage (NREM %)—Percentage of Non-Random Eye Movement Sleep of the TST
4. REM Latency—Time from onset of sleep to the first REM sleep stage
5. REM Density—Frequency of rapid eye movements during REM sleep
6. REM Quantity—Total amount of REM sleep (in minutes, or other appropriate units)
7. NREM Quantity—Total amount of NREM sleep (in minutes, or other appropriate units)
8. Length of each REM Period (in minutes, or other appropriate units)
9. Length of each Sleep Cycle (in minutes, or other appropriate units)
10. Delta Ratio—If using EEG, the ratio of slow wave activity between first and second NREM sleep cycle
11. REM Initiation Propensity—Number of occurrences of REM sleep stage initiation
12. Time for onset of sleep—How long it takes to fall asleep
13. Number of Awakenings—Number of non-provoked awakenings during sleep

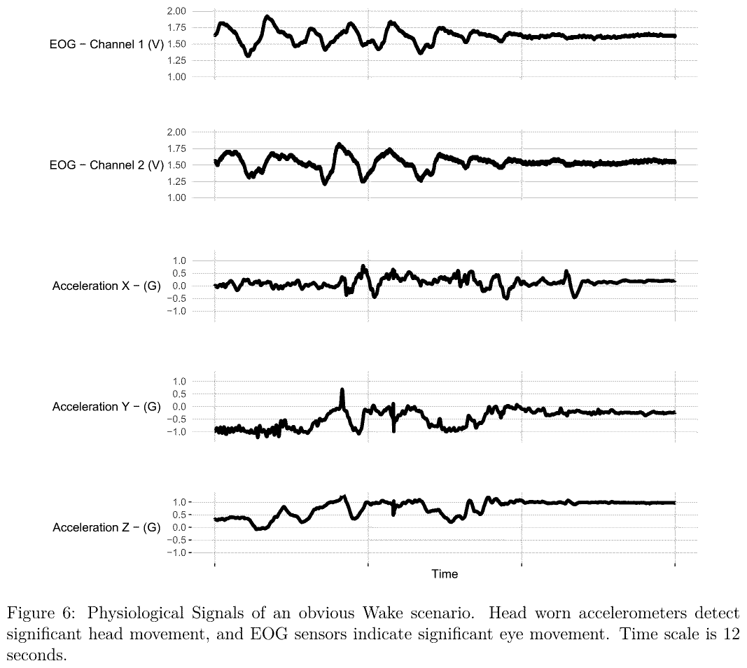

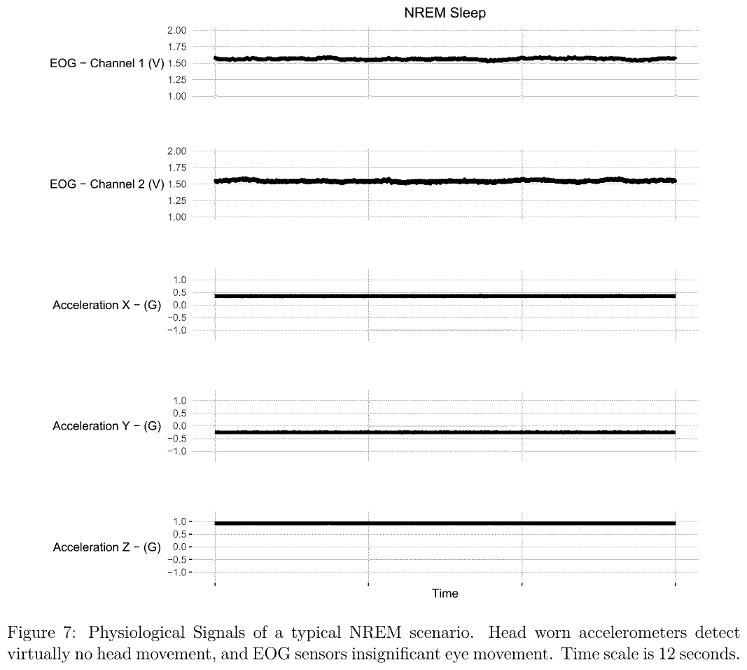

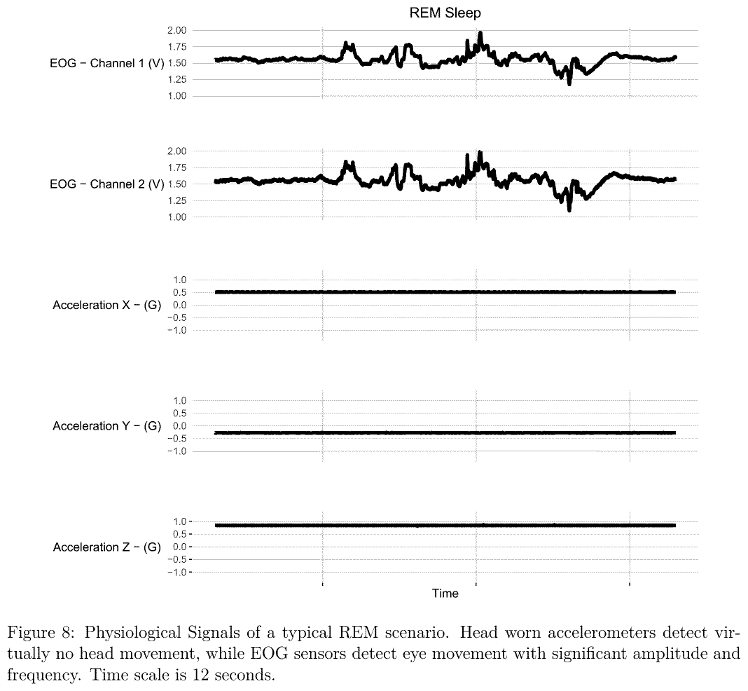

To briefly give an idea of what the various sleep stages may present as with various sensor types, we refer to FIGS. 6, 7, and 8. These plots show 5 sensor signals across a 12 second period for Wake, NREM sleep, and REM sleep respectively. The first two signals (EOG) show eye movement data, and that last 3 (actigraphy) show acceleration data of the head. FIG. 6 shows a clear Wake state where there is significant and frequent eye movements, shown in the EOG channels, and significant head movement, shown in all 3 accelerometer axes. FIG. 7 shows an NREM state where there is hardly any activity at all, only some minor fluctuations in the EOG signals. Finally, FIG. 8 shows frequent and significant eye movement, while there is no head movement.

Once the baseline period is completed, the next progression is to the treatment phase. During this period the substances of question will be administered to the appropriate subjects. In this example, subjects receive one daily dose of the substance for their respective group, for a duration of 5 days, as shown in FIG. 1. Explicitly, as seen in FIG. 4, all members of Group A, that is subjects A1, A2, . . . , AX, will receive Substance A. Similarly, subjects of Group B receive Substance B, and subjects of Group C receive Substance C. The Control group should not receive any experimental treatment, aside from a placebo. However, it is possible for a Control group to receive no placebo. And it is also possible to have an alternative Control group which receives a substance already known to have antidepressive properties and meet the 3 principal selection criteria described shortly.

Once the treatment period is accomplished, the next progression is to the recovery period. The treatment is discontinued, and similar to the previous two periods, the same sleep measures are collected. There should be no changes besides discontinuation of the treatment (and if used in the control, the placebo) during this time. It is noted that the recovery period is a short window after the treatment is discontinued. It is anticipated that any changes observed during treatment as caused by the experimental substances, will fade and sleep measures will eventually regress to those observed during the baseline period. This highlights the importance of examining sufficiently small observation windows, such as durations of 24 hours, in order to observe the trends that may be masked by longer period mean averages.

To clarify the time progression of the screening, we refer to FIG. 1. FIG. 1 presents the REM % during a 9 day trial which consist of 2 base line days (days 1-2), 5 treatment days (days 3-7), and 2 recovery days (days 8-9). We highlight that the timescales used in this figure and this example are chosen to best illustrate the concept of the method, not to serve as time frame recommendations. In humans, for example, the treatment time frame should be longer than 5 days in order to establish that the body does not readily habituate to the substance, which is one of the 3 principal selection criteria we will further describe later. For the sake of the example, we may assume that FIG. 1 shows the mean REM % of all individuals in Group C. The average REM % during the baseline is approximately 15%, while during treatment this drops to roughly 5%, and in recovery increases to a high of over 22%. This demonstrates that Substance C had a significant reductive effects on REM %, while maintaining or increasing the drive for REM sleep during the treatment phase.

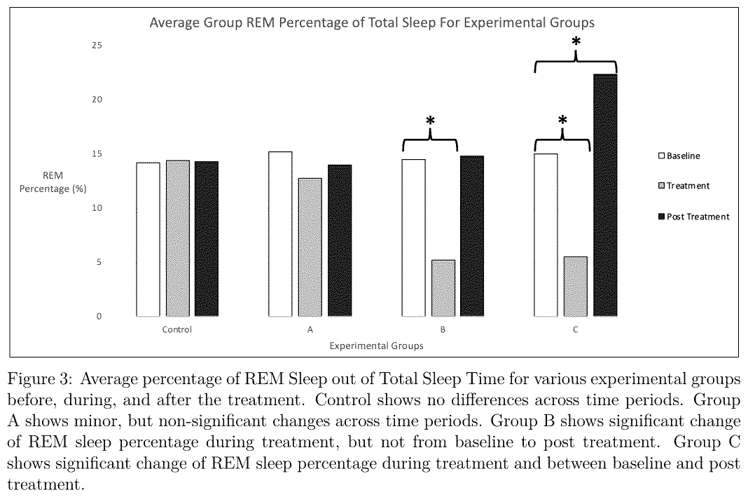

The graph in FIG. 3 shows what the results might look like across a screening with substances A, B, and C which each produce differing physiological effects. This graph presents the mean group REM % for baseline, treatment and recovery periods across the control and groups A, B, and C. As expected, the control group shows no changes in REM %. Substance A produced little change in REM % in Group A across the trial. Substance B had an effect on Group B, clearly reducing the REM % during the experimental period. However, the recovery period displays little difference in REM % with the baseline period, indicating that Substance B did not succeed in maintaining REM pressure. Finally, substance C both reduced REM % during the treatment period, and maintained or increased REM pressure during the treatment period, as evidenced by an obvious REM rebound in the recovery period, where the REM % is greater than that seen during the baseline period.

Considering the experimental set-up and information presented in both FIG. 1 and FIG. 3, it is possible to identify the substance which is a potential psychotropic candidate using the selection criteria in our method. The principal criteria for selection are:
1. A clear reduction in REM % during the treatment phase, compared with baseline
2. Persistent reduction in REM % during the treatment phase, such that no clear or significant habituation to the substance occurs
3. A clear REM rebound during the recovery phase, compared with baseline REM %

Based on these 3 principal criteria, only substance C is a potential candidate. Explicitly, substance C (1) caused a clear and obvious decrease in REM % during the treatment period compared with baseline, (2) did not present any signs of habituation by the subject during the treatment period as the reduction in REM % remains constant as observed in FIGS. 1, and (3) discontinuation of substance C resulted in a clear and obvious REM rebound in which the REM % during recovery was higher than baseline, indicating sustainment or elevation of REM pressure during treatment.

In addition to the 3 principal criteria, additional indicators are considered in the evalua-tion process to increase confidence in the selection choice or help select among multiple promising candidates. These indicators include:
1. An increase in the Delta Ratio
2. An increase in REM Density
3. An increase REM latency
4. Change in Ultradian Rhythm or Sleep Cycle Length

There may remain uncertainty after screening as to whether a given substance is worth selecting even if it satisfies the 3 principal criteria. This may be due to a substance either producing minimally sufficient changes, or on the other hand, if there are multiple substances that show promise but not all can be pursued for further testing. Using the additional indicators as denoted above will assist in this decision process. The Delta Ratio, or Delta Sleep Ratio, is the ratio of the slow wave activity during the first and second NREM period. An augmentation of this ratio during the treatment compared with that recorded during baseline is suggestive of a more promising candidate. Similarly, an increase during the treatment phase in the REM Density, the frequency of random eye movements during REM sleep, or an increase in the REM latency, the time to first enter REM sleep from sleep onset, would also be encouraging indicators for a given substance. Finally, a change to the ultradian rhythm or sleep cycle length is the last indicator. Ultradian rhythms are biological oscillations that occur on a daily cycle. During sleep, they reflect the alternation between NREM and REM sleep. Therefore it is possible that one or both change.

In the real world, economic and time pressures will be important factors to consider in the selection process. Multiple substances during a screening may appear promising, answering the requirements of the criteria and indicators, but only a few or even one can be selected for continued testing due to cost, time, and labor capacity. It is recommended that a prioritization ranking be implemented such that the substance with the highest ranking will be the first to undergo further testing.

The ranking process should be as follows: Once the experimental evaluation of the sub-stances (or substance) is complete, it must be determined to what extent they satisfy the 3 principal criteria. Substances which cause greater decreases of REM % during treatment, show little or no habituation, and demonstrate stronger REM rebound should generally be prioritized over drugs with weaker effects. It is important to take into consideration the additional indicators during this period as well, as a substance which may produce slightly weaker effects than another candidate ac-cording to the 3 principal criteria, may have noticeably stronger effects on the additional indicators, and therefore may be worth prioritizing in the selection process. If two substances produce identical effects, or nearly so, according to the 3 principal criteria, but differ in the additional indicators, then the indicators should inform the decision, everything else considered equal.

Other factors will also impact this ranking and selection process. These include, but are not limited to cost and difficulty in producing the substance, ease in administering the substance, and most importantly, side-effects caused by the substance observed during both the treatment and recovery period. With the goal of producing beneficial drugs, it is important to ensure that side-effects, especially those of a debilitating, painful, and serious nature should be avoided. Therefore, substances as they are during testing, which may qualify strongly according to the criteria and indicators, but causes significant or detrimental side-effects, should rank very poorly.

Once the ranking evaluation is complete, substances which ranked highly should be se-lected for further research, and if economic, time, and labor factors should allow in the future, the subsequently ranked substances should then be investigated.

We note here one additional method which can be incorporated into the above ranking selection or used separately for direct selection. As described earlier, an alternative Control group may be used which is administered a substance already known to have antidepressive properties and meets the 3 principal criteria. Example substances are imipramine, a TCA, or fluoxetine (Prozac), an SSRI. The observed effects during the screening can then be used as a selection threshold for the experimental substance(s), such that anything meeting or exceeding the threshold is considered for further testing.

It is finally noted that “significances” described in the text or shown in the figures high-light the differences between these example conditions, not that they are necessarily statistical significances and a prerequisite for determination of assay efficacy.

It will thus be seen that the objects set forth above, among those made apparent from the preceding descriptions, are efficiently attained and, because certain changes may be made in carrying out the above method and in the construction(s) set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the method herein described and all statements of the scope of the invention which, as a matter of language, might be said to fall therebetween.

### BIBLIOGRAPHY

Ira Albert, George A. Cicala, and Jerome Siegel. The behavioral effects of REM sleep deprivation in rats. Psychophysiology, 6(5):550-560, 1970. 4

Pierre M. Bet, Jacqueline G. Hugtenburg, Brenda W. J. H. Penninx, and Witte J. G. Hoogendijk. Side effects of antidepressants during long-term use in a naturalistic setting. European Neuropsy-chopharmacology, 23(11):1443-1451, 2013. 2

Debra J. Brody and Qiuping Gu. Antidepressant use among adults: United States, 2015-2018. US Department of Health and Human Services, Centers for Disease Control and Prevention, 2020. URL https://www.cdc.gov/nchs/products/databriefs/db377.htm. 3

Rosalind Cartwright, Erin Baehr, Jennifer Kirkby, S. R. Pandi-Perumal, and Julie Kabat. REM sleep reduction, mood regulation and remission in untreated depression. Psychiatry Research, 121(2):159-167, 2003. 4, 5

Cleveland Clinic. Serotonin syndrome: What it is, causes, symptoms & treatment, 2023. URL https://my.clevelandclinic.org/health/diseases/17687-serotonin-syndrome. Web Page, Cleveland Clinic. 2

John F Cryan and Andrew Holmes. The ascent of mouse: advances in modelling human depression and anxiety. Nature reviews Drug discovery, 4(9):775-790, 2005. 6

William Dement. The effect of dream deprivation: The need for a certain amount of dreaming each night is suggested by recent experiments. Science, 131(3415):1705-1707, 1960. 4

William Dement. Studies on the function of rapid eye movement (paradoxical) sleep in human subjects. Aspects Anatomo-Fonctionels de la Physiologie du Sommeil, 1965a. 4

William Dement and Charles Fisher. III. Experimental interference with the sleep cycle. Canadian Psychiatric Association Journal, 8(6):400-405, 1963. 4

William C. Dement. Experimental dream studies. Academy of Psychoanalysis: Science and Psy-choanalysis, 7:129-184, 1964. 4

William C. Dement. Recent studies on the biological role of rapid eye movement sleep. American Journal of Psychiatry, 122(4):404-408, 1965b. 4

D. L. F. Dunleavy, Vlasta Brezinova, Ian Oswald, A. W. Maclean, and M. Tinker. Changes during weeks in effects of tricyclic drugs on the human sleeping brain. The British Journal of Psychiatry, 120(559):663-672, 1972. doi: 10.1192/bjp.120.559.663. 5

Charles Fisher. Psychoanalytic implications of recent research on sleep and dreaming: Part I: Empirical findings. Journal of the American Psychoanalytic Association, 13(2):197-270, 1965. 4

David A. Flockhart. Dietary restrictions and drug interactions with monoamine oxidase inhibitors: an update. The Journal of clinical psychiatry, 73(suppl 1):4461, 2012. 3

Michael P. Hengartner. Methodological flaws, conflicts of interest, and scientific fallacies: Impli-cations for the evaluation of antidepressants' efficacy and harm. Frontiers in psychiatry, 8:275, 2017.

Mark Abie Horowitz and David Taylor. Tapering of SSRI treatment to mitigate withdrawal symp-toms. The Lancet Psychiatry, 6(6):538-546, 2019. 3

Anna Koski, Erkki Vuori, and Ilkka Ojanperä. Newer antidepressants: Evaluation of fatal toxicity index and interaction with alcohol based on Finnish postmortem data. International Journal of Legal Medicine, 119:344-348, 2005. 3

M. Levenson and C. Holland. Antidepressants and suicidality in adults: Statistical evaluation. Presentation at Psychopharmacologic Drugs Advisory Committee, 2006. 2

Aoibhin McCool, Kurt Lukas, Peter Hayes, and Dervla Kelly. Antidepressant medication prescribing patterns in Irish general practice from 2016 to 2020 to assess for long-term use. Irish Journal of Medical Science (1971-), pages 1-8, 2021. 3

Patrick McNamara. The Neuroscience of Sleep and Dreams. Cambridge Fundamentals of Neu-roscience in Psychology. Cambridge University Press, 2019. ISBN 9781107171107; 1107171105. 4

Wallace B. Mendelson, Robert D. Guthrie, Ghislaine Frederick, and Richard Jed Wyatt. The flower pot technique of rapid eye movement (REM) sleep deprivation. Pharmacology Biochemistry and Behavior, 2(4):553-556, 1974. 4

David B. Menkes and Andrew Herxheimer. Interaction between antidepressants and alcohol: Signal amplification by multiple case reports. International Journal of Risk & Safety in Medicine, 26(3):163-170, 2014. 3

Joanna Moncrieff, Ruth E. Cooper, Tom Stockmann, Simone Amendola, Michael P. Hengartner, and Mark A. Horowitz. The serotonin theory of depression: a systematic umbrella review of the evidence. Molecular psychiatry, pages 1-14, 2022. 6

Bruce Morden, George Mitchell, and William Dement. Selective REM sleep deprivation and com-pensation phenomena in the rat. Brain research, 5(3):339-349, 1967. 4

James W. Nielsen, Malgorzata W. Witek, and Stephen Hurwitz. Optimal antidepressant dosing: Practical framework for selection, titration, and duration of therapy. Postgraduate Medicine, 108(5):111-115, 2000. 2

OECD. Pharmaceutical market: Pharmaceutical consumption: N06a-antidepressants, 2023. URL https://stats.oecd.org/Index.aspx?ThemeTreeId=9#. Web Page, Organization for Economic Co-operation and Development (OECD) Statistics. 3

Solomon S. Steiner and Steven J. Ellman. Relation between REM sleep and intracranial self-stimulation. Science, 177(4054):1122-1124, 1972. 4

Warren C. Stern, Francis P. Miller, Raymond H. Cox, and Roger P. Maickel. Brain norepinephrine and serotonin levels following REM sleep deprivation in the rat. Psychopharmacologia, 22:50-55, 1971. 4

Rebecca Strawbridge, Allan H. Young, and Anthony J. Cleare. Biomarkers for depression: recent insights, current challenges and future prospects. Neuropsychiatric disease and treatment, pages 1245-1262, 2017. 3

Rudolf Uher, Anne Farmer, Neven Henigsberg, Marcella Rietschel, Ole Mors, Wolfgang Maier, Dejan Kozel, Joanna Hauser, Daniel Souery, Anna Placentino, et al. Adverse reactions to antide-pressants. The British Journal of Psychiatry, 195(3):202-210, 2009. 2

Gerald Vogel and Arthur Traub. Further studies on REM deprivation of depressed patients. Psy-chophysiology, 1968a. 4, 5, 6

Gerald W. Vogel. A review of REM sleep deprivation. Archives of General Psychiatry, 32(6):749-761, 1975. doi: 10.1001/archpsyc.1975.01760240077006. 4

Gerald W. Vogel. Evidence for REM sleep deprivation as the mechanism of action of antidepressant drugs. Progress in Neuro-Psychopharmacology & Biological Psychiatry, 1983. 5

Gerald W. Vogel and Arthur C. Traub. REM deprivation: I. the effect on schizophrenic patients. Archives of General Psychiatry, 18(3):287-300, 1968b. 4, 5

Gerald W. Vogel, Arthur C. Traub, Pessah Ben-Horin, and George M. Meyers. REM deprivation: II. The effects on depressed patients. Archives of General Psychiatry, 18(3):301-311, 1968. 4, 5, 6

Gerald W. Vogel, Arthur Thurmond, Pauline Gibbons, Katherine Sloan, Mary Boyd, and Margaret Walker. REM sleep reduction effects on depression syndromes. Archives of General Psychiatry, 32(6):765-777, 1975. doi: 10.1001/archpsyc.1975.01760240093007. 5

Gerald W. Vogel, A. Buffenstein, K. Minter, and Ann Hennessey. Drug effects on REM sleep and on endogenous depression. Neuroscience & Biobehavioral Reviews, 14(1):49-63, 1990. doi: 10.1016/S0149-7634(05)80159-9. 5, 6

Paul Willner. Validity, reliability and utility of the chronic mild stress model of depression: a 10-year review and evaluation. Psychopharmacology, 134:319-329, 1997. 6

World Health Organization. Depression and other common mental disorders: Global health esti-mates. Technical report, World Health Organization (WHO), 2017. 2

### List of Figures

1 Percentage of REM Sleep out of Total Sleep Time over several days. ‘BX’ denotes Baseline day, ‘TX’ denotes Treatment day, ‘PX’ denotes Post-treatment day. Average baseline REM sleep percentage is ˜15%, treatment causes a significant reduction in REM percentage lowering it to ˜5%, and there is a post-treatment rebound up to ˜22%.

2 Some methods for observation of test animals

3 Average percentage of REM Sleep out of Total Sleep Time for various experimental groups before, during, and after the treatment. Control shows no differences across time periods. Group A shows minor, but non-significant changes across time periods. Group B shows significant change of REM sleep percentage during treatment, but not from baseline to post treatment. Group C shows significant change of REM sleep percentage during treatment and between baseline and post treatment.

4 Example screening of a homogenous rodent population divided into three testing groups, A, B, and C. Each group contains N=1, 2, . . . , X testing animals as denoted in the figure. In this example, a rodent with contact sensors [16] is presented as the animal model and method of monitoring, with each animals having its own enclosure [19]. Each animal is labeled first by their respective group letter, i.e., A, B, or C, followed by respective number in sequential order, i.e., 1, 2, . . . , X

5 Method of blocking epochs for real time classification.

6 Physiological Signals of an obvious Wake scenario. Head worn accelerometers detect significant head movement, and EOG sensors indicate significant eye movement. Time scale is 12 seconds.

7 Physiological Signals of a typical NREM scenario. Head worn accelerometers detect virtually no head movement, and EOG sensors insignificant eye movement. Time scale is 12 seconds.

8 Physiological Signals of a typical REM scenario. Head worn accelerometers detect vir-tually no head movement, while EOG sensors detect eye movement with significant amplitude and frequency. Time scale is 12 seconds.

What is claimed:

1. A method for screening potential therapeutic substances, comprising: (a) one or more animal subjects or human participants; (b) sleep measurements of the entities described in (a) during a baseline period; (c) administration of the prospective substance(s) during a testing period; (d) sleep measurements of the entities described in (a) during the testing period; (d) discon-tinuation of the prospective substance(s); (e) sleep measurements of the entities described in (a) during the recovery period; and (f) evaluation of changes and trends between and within the sleep measurements during baseline, treatment, and recovery periods.

2. The method of claim 1 wherein sleep measurement methods, either direct or correlative, may include but are not limited to one or a combination of: Electroencephalography (EEG), Near-Infrared Spectroscopy (NIRS), Electrooculography (EOG), Electromyography (EMG), Heart Rate (HR), Respiration Rate, Actigraphy from any part of the body, In-Vivo Electrophysio-logical recordings such as Single- and Multiple-Unit Recording, Optogenetics, and non-contact measurement methods such as video and radar.

3. The method of claim 1 wherein the prospective substance may be a single compound or a mixture of compounds.

4. The method of claim 1 wherein the prospective substance may also denote non-pharmacological interventions, including physical methods such as, but not limited to, stimulation with lights, electricity, and magnetic fields.

5. The method of claim 1 wherein administration encompasses variable dosage frequencies such as but not limited to once per hour, once per day, once per week, etc.

6. The method of claim 1 wherein the baseline period denotes a period during screening where animal models or human participants are monitored to establish typical, ecological measures, and there is a refrain from any external variables which could affect the measures.

7. The method of claim 1 wherein treatment period denotes a period during screening where an-imal models or human participants are exposed to the experimental substance(s) in question.

8. The method of claim 1 wherein the recovery period, otherwise known as the post treatment period, denotes a period during screening where animal models or human participants have previously been exposed to the substance(s) in question, and that exposure has been discon-tinued. During the recovery period there is a refrain from any external variables that could affect the sleep measures.

9. The method of claim 1 wherein the evaluation of changes between screening periods of baseline, treatment, and recovery must meet 3 criteria, comprising: (a) an obvious decrease in REM % during treatment, as evidenced by consideration of the REM % difference between baseline and treatment %; (b) an obvious REM rebound post treatment, as evidenced by consideration of the REM % difference between baseline and recovery, such that there is an increase in the latter; and (c) should any decrease in REM % occur during treatment, an obvious resistance to habituation to this decrease exists.

10. The method of claim 1 wherein the evaluation of changes between and within screening periods may include as further evidence for a successful candidate, wherein: (a) the REM density level, the frequency of eye movements during REM sleep, observed at baseline increases during the treatment period; (b) the Delta ratio, the ratio of slow wave activity between the first and second NREM cycles, increases during the treatment period as compared to baseline; (c) REM latency, the time for onset for the first REM cycle after falling asleep, as measured during baseline, increases during treatment, meaning it takes longer to enter REM sleep, and (d) changes to the ultradian rhythm or sleep cycle length.
